(19) United States
(12) Patent Application Publication
(10) Pub. No.: US 2008/0262535 A1
(43) Pub. Date: Oct. 23, 2008

(54) Method and an apparatus for adjusting Blood circulation in a limb

(75) Inventors: Noam Gavriely, Haifa (IL); Oren Gavriely, Haifa (IL)

Correspondence Address: Martin D. Moynihan;PRTSI, Inc., P.O. Box 16446, Arlington, VA, 22215, US

(73) Assignee: OHK Medical Devices Ltd., Haifa (IL)

(21) Appl. No.: 12/078,785

(22) Filed: Apr. 4, 2008

Related U.S. Application Data
(60) Provisional application No. 60/922,030, filed on Apr. 6, 2007.

(51) Int. Cl. A61B 17/132

(52) U.S. Cl. 606/203

(57) ABSTRACT

A mechanized tourniquet adapted for application in a surgery, emergency or military situation to stop arterial blood loss in an injured limb, which is optionally activated by a user or automatically by detecting therein the free tip of its pressure-applying band. The tourniquet and its electromechanical tension device are automatically controlled by means of the band tension controlling mechanism and a timer initialized by a safety signal.

### RELATED APPLICATIONS

This Application claims the benefit of U.S. Provisional Patent Application No. 60/922,030, filed on Apr. 6, 2007, the contents of which are hereby incorporated in its entirety by reference.

### FIELD AND BACKGROUND OF THE PRESENT INVENTION

The present invention, in some embodiments thereof, relates to medical devices, optionally for blocking of blood flow. More particularly, but not exclusively, the present invention relates to the devices and methods for use in surgery and in emergency and military medicine.

Arterial tourniquets have been used in surgery, emergency and military medicine for over 300 years for emergency treatment of bleeding or injury of human limbs (see U.S. Pat. No. 34,112; U.S. Pat. No. 35,038). The arterial tourniquets are used to occlude the blood from flowing in the arteries into a limb.

Tourniquets with means for regulating applied pressure are disclosed, for example, in U.S. Pat. No. 5,048,536; U.S. Pat. No. 5,181,522 and U.S. Pat. No. 6,682,547. U.S. Pat. No. 6,682,547 disclose a tourniquet of the Zimmer type, which is supplied by an identification means for indicating of the tourniquet's physical characteristics.

U.S. Pat. No. 6,746,470 and US Patent Application No. 2003/0139766 “Emergency and Military Tourniquet for Pre-Hospital Use” disclose a pneumatic tourniquet adapted for self application by an injured person in a military or emergency situation to stop blood loss from an injured limb. A pneumatic tourniquet comprises a bladder cuff with a clamp means for securing the bladder around the limb and an indicator module connected to the bladder. The indicator module indicates the cuff pressure and, in some instances, the elapsed inflation time interval up to 2 hours and is supplied with a microprocessor and an alarm indicator that provides an audible and a visual indication of an alarm to the crew or the user. The microprocessor is programmed to determine elapsed inflation time by measuring the duration of time that the pressure has exceeded a predetermined pressure threshold. The alarm indicator may also be activated by microprocessor if unusually high pressures are detected in the bladder (for example pressures greater than 400 mm/Hg).

Self-regulating pneumatic tourniquets with a pressure application source are described in US Patent No. 2007/0191881 and in GB Patent No. 713132. A tourniquet is supplied by an aneroid actuator and the timing spring driving mechanism.

U.S. Pat. No. 4,243,039 discloses an emergency tourniquet, destined to be used on the battlefield, comprises a strap and a combined mechanism for tightening the strap and indicating the pressure exerted on the limb. The mechanism consists of a casing that contains a spool on which the free end of the strap is wound up by means of a pawl and a tension-indicating device.

Tourniquets with the tension mechanisms are described in U.S. Pat. No. 1,279,784; US Patent Application No. 2006/0025807, in US Patent Application No. 2005/0113866 and in US Patent Application No. 2005/0267518. A tourniquet includes at least one gear mechanism with a worm shaft and a worm wheel. Other embodiments of mechanically driven tourniquets are described in U.S. Pat. No. 3,095,873 and in U.S. Pat. No. 2,604,098.

### SUMMARY OF THE PRESENT INVENTION

According to some aspects of the present invention there is provided an apparatus for adjusting blood circulation in a limb. The apparatus comprises a pressure applying band, having a central longitudinal axis and parallel lateral edges, configured for circumferentially wrapping a portion of the limb and applying a first circumferential pressure beneath the parallel lateral edges and a second circumferential pressure beneath proximity of the central longitudinal axis. The first circumferential pressure is lower than the second circumferential pressure.

Optionally, the pressure applying band having a non linear cross sectional configuration.

More optionally, the non linear cross sectional configuration having a shape selected from a group consisting of a crescent shape, a semi-parabolic shape, a semi-elliptic shape, an elliptic shape, a parabolic shape, or a semicircular shape.

More optionally, the non-linear cross sectional configuration is fixed.

Optionally, the proximity of the central longitudinal axis has a first elasticity coefficient and the parallel lateral edges having a second elasticity coefficient. The first elasticity coefficient is higher than the second elasticity coefficient.

More optionally, the apparatus further comprises a friction reduction layer between the pressure applying band and the limb, the friction reduction being configured for reducing the friction between the limb and the pressure-applying band.

Optionally, the apparatus further comprises an automatic traction mechanism configured for pulling the pressure applying band to encircle tightly the limb.

More optionally, the apparatus further comprises a pressure adjusting module configured for adjusting the first and second circumferential pressures by activating the traction mechanism according to at least one of a predefined pressure level, a user input defining a pressure level.

More optionally, the apparatus further comprises a pressure adjusting module and at least one pressure sensor configured for measuring at least the second circumferential pressure, the pressure adjusting module being configured for activating the automatic traction mechanism according to the measured second circumferential pressure.

Optionally, the apparatus further comprises a manual traction mechanism configured for allowing a user to pull the pressure applying band around the limb.

According to some aspects of the present invention there is provided an apparatus for adjusting blood circulation in a limb. The apparatus comprises a pressure applying band for being wrapped around the limb to apply a circumferential pressure thereon, a traction mechanism configured for pulling the pressure applying band tightly around the limb, at least one sensor configured for measuring the circumferential pressure, and a pressure adjusting module configured for activating the traction mechanism according to the measured applied pressure for adjusting the circumferential pressure.

Optionally, the apparatus further comprises a presentation unit for presenting the measured circumferential pressure.

Optionally, the apparatus further comprises a user interface for allowing a user to input a requested circumferential pressure, the pressure adjusting module being configured for activating the traction mechanism according to the requested circumferential pressure.

Optionally, the at least one sensor comprises at least one of a pressure detection element for measuring the circumferential pressure and a tension detection element for detection a tension level of the pressure applying band.

More optionally, the pressure detection element is a member of a group consisting of a linear variable differential transformer (LVDT), a linear potentiometer, and a pressure transducer.

Optionally, the apparatus further comprises a timer module configured for timing a period during which the pressure applying band applies the circumferential pressure around the limb.

Optionally, the apparatus further comprises an alarming unit for alarming a user according to the timed period.

Optionally, the apparatus further comprises a memory element for storing a plurality of pulling patterns, the user interface being configured for allowing the user to select one of the plurality of pulling patterns, the requested circumferential pressure being adjusted according to the selected pulling pattern.

More optionally, each one of the pulling pattern is defined according to medical information of a patient, the medical information being selected from a group consisting of an age group, a gender, a medical condition, a blood pressure, patient history, and sensitivities.

Optionally, the traction mechanism having a feeding opening for supporting a positioning of the pressure applying band to allow the pulling, further comprising a band detection sensor for detecting a presence of the pressure applying band in a proximity to the feeding opening, the pressure adjusting module being configured for activating the traction mechanism according to the detection.

More optionally, the pressure applying band has a corrugated tip for facilitating the pulling.

Optionally, the apparatus further comprises a communication unit for transmitting the measured circumferential pressure to a remote computing unit.

Optionally, the apparatus further comprises a memory element for recording the period.

Optionally, the traction mechanism comprises at least one traction gearwheel, the traction mechanism being configured for actuating the at least one traction gearwheel for allowing the pulling, the pressure applying band having at least one groove for increasing the friction between the at least one traction gearwheel and the pressure applying band.

Optionally, the apparatus further comprises a quick-release mechanism configured for allowing a prompt reduction of the circumferential pressure.

According to some embodiments of the present invention there is provided a method for adjusting blood circulation in a limb. The method comprises providing a required circumferential pressure, allowing a user to encircle a pressure applying band around a limb, detecting the encircling, and mechanically adjusting the pressure applying band to apply the required circumferential pressure around the limb.

Optionally, the mechanically adjusting comprises pulling the pressure applying band while sensing the circumferential pressure. The pulling is stopped according to a ratio between the sensed circumferential pressure and the required circumferential pressure.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the present invention are herein described, by way of example only, with reference to the accompanying drawings. With specific reference now to the drawings in detail, it is stressed that the particulars shown are by way of example and for purposes of illustrative discussion of embodiments of the present invention. In this regard, the description taken with the drawings makes apparent to those skilled in the art how embodiments of the present invention may be practiced.

In the drawings:

FIG. 1 is a schematic illustration of a circulation control apparatus that is drawn tightly around a limb, according to some embodiments of the present invention;

FIG. 2 is a sectional schematic illustration of a crescent shape pressure-applying band 102, in accordance with some embodiments of the present invention;

FIG. 3 is a sectional schematic illustration of a segment of the pressure-applying band that is depicted in FIG. 1, according to some embodiments of the present invention;

FIG. 4 is a sectional schematic illustration of a circulation control apparatus, according to some embodiments of the present invention;

FIG. 5 is a schematic illustration of a circulation control apparatus with a set of pressure sensors, according to some embodiments of the present invention;

FIGS. 6-7 depict crosscuts of the schematic illustration of the FIG. 5, according to some embodiments of the present invention;

FIG. 8 depicts a sectional schematic illustration of a pressure-applying band that is cut in the middle for creating a niche for a pressure sensor, according to some embodiments of the present invention;

FIG. 9 is a schematic illustration of the pressure-applying band and tension unit of FIG. 4 from the top, according to some embodiments of the present invention;

FIG. 10 is a sectional schematic illustration of a tension unit and a pressure-applying band, which are depicted in FIG. 9, according to some embodiments of the present invention;

FIG. 11 is a sectional schematic illustration of gears and ratchets of exemplary traction mechanism 340, according to some embodiments of the present invention;

FIGS. 12-14 are crosscuts of transmission segments of the traction mechanism that is depicted in FIG. 11, according to some embodiments of the present invention;

FIG. 15 is a schematic illustration of a section of the pressure-applying band from above, according to some embodiments of the present invention;

FIG. 16 is a schematic illustration of a crosscut of the pressure-applying band of FIG. 15, according to some embodiments of the present invention; and

FIG. 17 is a schematic illustration of a method for controlling the flow of blood of arteries and/or veins of a limb, according to some embodiments of the present invention.

### DESCRIPTION OF EMBODIMENTS OF THE PRESENT INVENTION

The present invention, in some embodiments thereof, relates to medical devices and methods for using medical devices. More particularly, but not exclusively, the present invention relates to the devices and methods for controlling venous and arterial circulation in a limb.

According to some embodiments of the present invention, there is provided a pressure control apparatus for controlling venous and arterial circulation in a limb. The pressure control apparatus includes a pressure-applying band having a cross-sectional profile for applying a graded pressure on the limb along the latitudinal axis of the pressure-applying band and/or along one or more longitudinal axis of the pressure-applying band. Optionally, the cross-sectional profile of the pressure-applying band along the latitudinal axis and/or of each one of the strips of the pressure-applying band has a parabolic, elliptic, semi-elliptic, or semicircular shape. The cross-sectional profile is designed to apply graded pressure in a manner that the applied pressure intensified toward the central longitudinal axis of the pressure-applying band. Optionally, the pressure-applying band has an inconstant elasticity along its longitudinal axis. In an exemplary embodiment of the present invention, the pressure-applying band is thicker and/or has a lower elasticity coefficient in the middle of its latitudinal axis than in its lateral edges. Optionally, such an elasticity and/or variable thickness reduce the pressure on the tissues during the usage of the pressure-applying band in order to avoid and/or to reduce damaging nerves.

According to an aspect of some embodiments of the present invention there is provided a pressure control apparatus that includes a pressure-applying band and a tension unit. In use, the tension unit, which may be a mechanical or an electromechanical tension unit, is mounted on or around the pressure-applying band that is wraparound or substantially wraparound a limb. The tension unit is designed to adjust the pressure that is applied by the pressure-applying band on the limb by putting and/or releasing the pressure-applying band.

Optionally, the pressure-applying band has a number of pressure-applying strips along its longitudinal axis. Each strip is optionally thicker and/or has a lower elasticity coefficient in the middle thereof than in its lateral edges.

Optionally, the pressure-applying band has a friction reduction layer for reducing the friction between the limb and the pressure-applying band. The layer reduces the friction that is caused by the traction that is applied during the drawing of the pressure-applying band tightly around the limb. Optionally, the friction reduction layer comprises a sheet of thin and low-friction material, such as silk and/or any other smooth synthetic fabric.

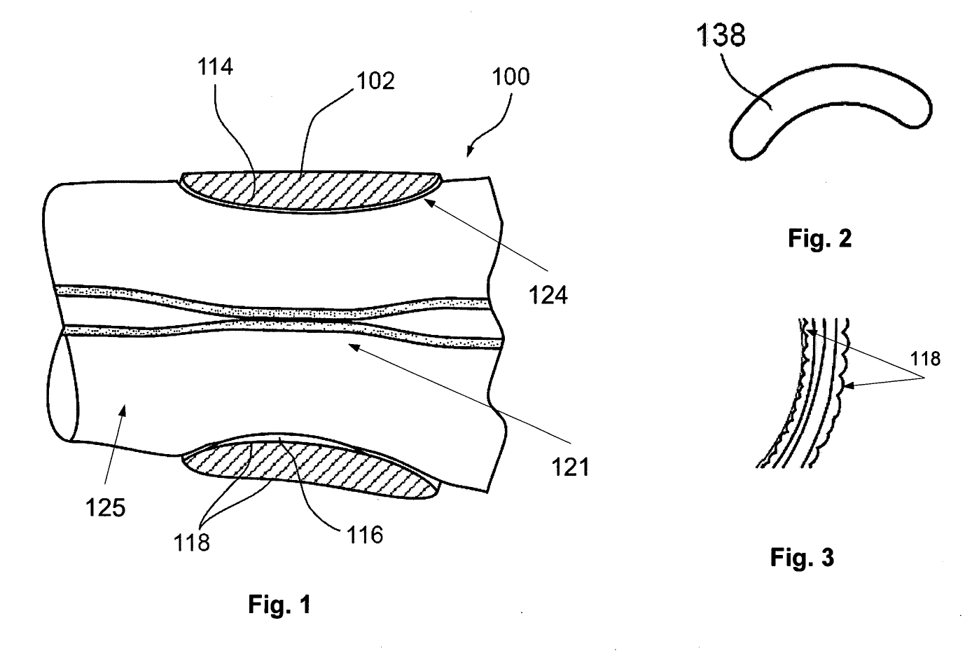

Reference is now made to FIG. 1, which is a schematic illustration of a circulation control apparatus 100 that is drawn tightly around a limb, according to some embodiments of the present invention. The circulation control apparatus 100 includes a pressure-applying band 102 that is designed for applying circumferential pressure upon the skin and underlying tissues of a limb, such as a hand or a leg. This pressure is transferred to the walls of vessels in the limb, causing them to become temporarily occluded, or substantially occluded, for example as shown at 121. The pressure-applying band 102 has a cross-sectional profile that is designed for allowing it to apply graded pressure on the encircled limb.

Optionally, the cross-sectional profile along the latitudinal axis of the pressure-applying band 102 is thicker in its middle than at around its lateral edges, for example as shown at 124. Such a cross-sectional profile may have a semicircular shape, a semi-elliptic shape, a shape that is generally thick in its middle and becomes thinner towards its edges, or any other shape that allows the distribution of pressure as described below. Such a sectional profile along the longitudinal axis of the pressure-applying band 102 assures that the pressure-applying band 102 applies circumferential pressure that gradually increases, optionally linearly, toward an area of the limb that is encircled by the central longitudinal axis of the pressure-applying band 102, for example as shown at FIG. 1.

Optionally, the pressure-applying band 102 has an inconsistent elasticity coefficient along the latitudinal and/or longitudinal axes thereof. In such a manner, the pressure that is applied on the limb that is encircled by the pressure-applying band 102 is spread in unevenly along the latitudinal and/or longitudinal axes, creating a higher pressure in the center of the pressure-applying band 102 than one the lateral edges thereof.

In use, the pressure-applying band 102, which may be drawn tightly around the limb as a commonly known tourniquet, applies a circumferential pressure upon the skin and underlying tissues of a certain limb. For clarity, a circumferential pressure means a pressure that is circumferentially applied by the pressure-applying band 102 on the tissues which encircled beneath it.

However, as the pressure it applies gradually increases toward the area of the limb that is beneath the area in a proximity to the central longitudinal axis of the pressure-applying band 102, the pressure that is transferred to the walls of vessels of the limb is applied gradually for example as shown at 124 of FIG. 1. The pressure of the pressure-applying band 102 is spread along the vessels which are located beneath it and not directly applied on one or more direct pressure point along the vessel. Furthermore, the compression of the tissues of the blood vessels and nerves beneath the edges of the pressure-applying band 102 is reduced.

In such a manner, the damage that may be caused to tissues, such as nerves, at such direct pressure points is avoided and the damage that is caused to the limb by the occlusion may be reduced.

Optionally, the cross-sectional profile of the pressure-applying band 102 has a crescent shape, for example as shown at FIG. 2, which is a sectional schematic illustration of the pressure-applying band 102, according to some embodiment of the present embodiment. In such an embodiment, the pressure-applying band 102 is optionally made of a smart material that maintains a crescent shape 138 of the cross-sectional profile.

In some embodiments of the present invention, the pressure-applying band 102 is substantially wider than the area on which it applies most of its pressure. In such a manner, the damage to tissues, which are in the proximity of the occlusion and/or substantial occlusion of the limb, is reduced. Furthermore, the aforementioned cross-sectional shape minimizes the shear stress that is applied on the area in the proximity of the edges of the pressure-applying band 102 and reduces the damage to blood vessels and nerves that is caused by redundant occlusion.

The longitudinal dimension of the pressure-applying band 102 ranges from at least 14 centimeters, a length that allows using the circulation control apparatus 100 for creating an occlusion in an arm of a child, to more than 100 centimeters, a length that allows using the circulation control apparatus 100 for creating an occlusion in a thigh of a large individual. Optionally, the latitudinal dimension of the pressure-applying band 102 ranges from at least 4 centimeters to about 12 centimeters and the vertical dimension of the pressure-applying band 102 ranges from at least 0.4 centimeters to 1.5 centimeters or more.

Reference is now also made to FIG. 3, which is a sectional schematic illustration of a segment of the pressure-applying band 102 that is depicted in FIG. 1, according to some embodiments of the present invention. Optionally, the pressure-applying band 102 is encircled with and/or attached to a friction reduction layer 118. The friction reduction layer 118 is designed to protect the skin of the encircled limb, for example the limb that is shown at 125, from the pressure that is applied circumferentially thereon by the tension of the pressure-applying band 102. Optionally, the friction reduction layer 118 includes a sheet of thin material with low friction coefficient, such as silk and/or synthetic fabric having a similar smoothness.

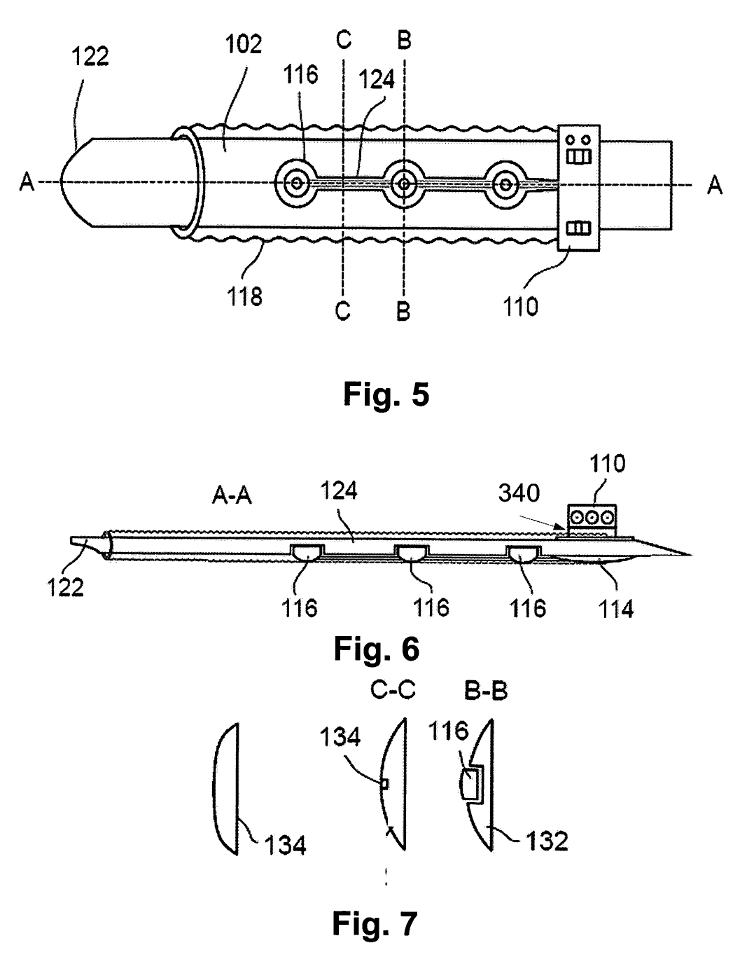

Optionally, the friction reduction layer 118 is shaped as a sleeve that encircles the pressure-applying band 102, for example as shown at FIGS. 5 and 6. The friction reduction layer 118 may be used for reducing and/or eliminating the damages caused by the sheer stress of pressure that is applied by the circulation control apparatus 100.

Optionally, a manual tension unit is used in order to support the pulling of the pressure-applying band 102 tightly around the limb. Such a manual tension unit allows the patient and/or the caretaker to pull the pressure-applying band 102 to until a desired pressure is applied on the limb that is encircled by the pressure-applying band 102. Such manual tension units are generally well known in the art and are, therefore, not described herein greater detail.

Optionally, an automatic tension unit is used in order to support the pulling of the pressure-applying band 102 tightly around the limb. A circulation control apparatus 100 that includes such a automatic tension unit is described, among other embodiments, hereinbelow.

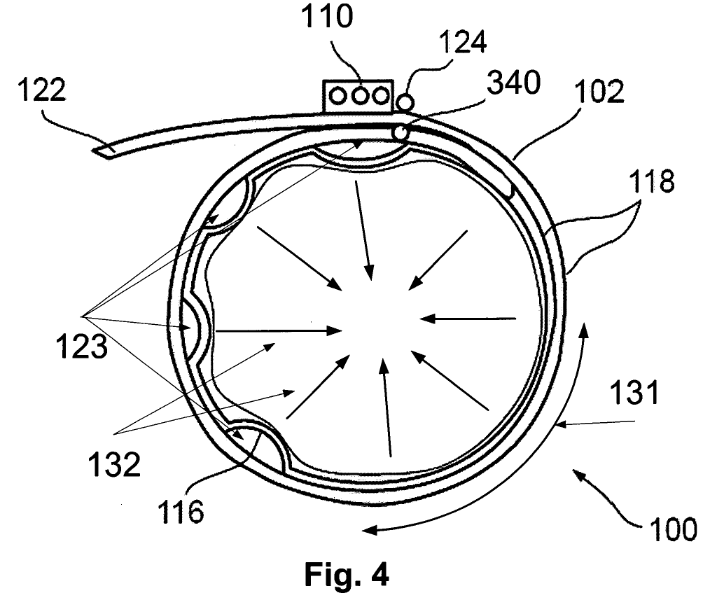

Reference is now also made to FIG. 4, which is a sectional schematic illustration of the circulation control apparatus 100, according to some embodiments of the present invention. FIG. 4 depicts a pressure-applying band, which may be the pressure-applying band 102 of FIG. 1, or any other pressure-applying band. FIG. 4 further depicts a tension unit 110, optionally automatic, for controlling the pressure that is applied on a limb by pressure-applying band 102. In use, the pressure-applying band 102 is drawn around a limb and the tension unit 110 is designed to pull and/or to release the pressure-applying band 102 for adjusting the amount of pressure that is applied on the limb, for example as further described below.

As the tension unit 110 may be used for pulling and/or releasing the pressure-applying band 102, a patient and/or a caretaker may use only one hand for operating to the circulation control apparatus 100 for controlling venous and arterial circulation of a limb and/or a portion of a limb. As used herein, a caretaker means an unskilled caretaker, a physician, a nurse, a medical assistant, and/or the like.

As further described below, after the patient and/or the caretaker brings the tip 122 of the pressure-applying band 102 to a feeding opening in the circulation control apparatus 100. The circulation control apparatus 100 pulls, optionally automatically, the pressure-applying band 102 until it is drawn tightly around the limb. This action applies circumferential pressure on the skin and the underlying tissues of the limb. This pressure is transferred to the walls of vessels, causing them to become temporarily occluded and allows the patient and/or the caretaker to slow or to prevent blood loss which may be caused by an injury and/or to prepare the limb to a medical operation.

The circulation control apparatus 100 may be used for reducing the period that is needed for taking care of wounded, allowing a wounded and/or unskilled patient and/or caretaker to occlude blood flow from an injury without causing, or substantially without causing, damage to the occluded limb.

As further described below, the tension unit 110 is connected to one or more sensors that measure the pressure that is applied by the circulation control apparatus 100. These measurements may be used for pulling and/or releasing the pressure-applying band 102, thereby to control the amount of pressure that is applied on the limb. In such a manner, using the circulation control apparatus 100 may reduce damages which are caused to tissues beneath and/or in the proximity of the pressure-applying band 102. As further described below, the tension unit 110 is connected to a presentation unit, such as a display and/or a speaker. The presentation unit may be used for generating an alarm and/or a set of instructions, which may assist the patient and/or the caretaker during the occlusion process.

Optionally, the pressure-applying band has a number of pressure-applying projections 123 along its longitudinal axis. Each projection 123 is optionally thicker and/or has a lower elasticity coefficient in the middle than in its lateral edges.

As further described below, each one of the projections 123 may include a sensor 116, such as a pressure sensor and/or a tension sensor, that is connected to a controller at the tension unit 110, which may be referred to herein as a pressure evaluating module (not shown). As commonly known, according to the Law of Laplace, the tension 131 of the pressure-applying band 102 may be converted to the pressure 132 that is applied by the pressure-applying band 102 on the limb. The larger the vessel radius, the larger the wall tension required to withstand a given internal fluid pressure. For a given vessel radius and internal pressure, a spherical vessel will have half the wall tension of a cylindrical vessel. For brevity, both a pressure sensor and/or a tension sensor are commutatively referred to as a pressure sensor.

Optionally, the pressure evaluating module manages the communication between modules, which are locally hosted in the tension unit 110, such as the pressure sensors and the user interface that is described below, and/or the communication between the tension unit 110 and devices and/or units which are external thereto, optionally using the communication unit, as described below. The pressure evaluating module may process the measurements which are gathered by the pressure sensors.

Reference in now also made to FIGS. 5-7, which are schematic illustration of the circulation control apparatus 100, according to some embodiments of the present invention. FIGS. 5 and 6 depict the circulation control apparatus 100 of FIG. 4 when it is stretched along its longitudinal axis. FIG. 6 depicts a crosscut of the circulation control apparatus 100 along the longitudinal axis, which is denoted with the letter A. FIG. 7 depicts crosscuts of the circulation control apparatus 100, which are stretched along axes C and B of the circulation control apparatus 100 as depicted in FIG. 5. As described above, a sleeve of friction reduction layer 118 covers the pressure-applying band 102 and reduces the damages which may be caused by the sheer stress that is applied by the pulling of the pressure-applying band 102 around the limb.

Optionally, the pressure sensor 116 comprises one or more individual spring loaded displacement sensors, such as linear variable differential transformers (LVDT), for example Sensotec S3C DC-DC Miniature of Honeywell™, which the specification thereof is incorporated herein by reference, or linear potentiometers, or may be sealed chambers connected with a tube to a pressure transducer such as a solid state miniature single sided or differential pressure transducer, for example PX138-001D5V, which the specification thereof is enclosed herein by reference. In such an embodiment, the pressure evaluating module is used for using the measurements of the pressure sensors for calculating the pressure that is applied by the pressure-applying band 102 during the pulling thereof around the limb it encircles.

Optionally, the pressure sensor 116 is a tension sensor that comprises a load cell or any other electronic device, such as a transducer, that is used to convert force into an electrical signal. In such an embodiment, the pressure evaluating module is used for calculating the pressure that is applied on the limb according to the measurements of the tension sensors that indicate the tension of the pressure-applying band 102 during the pulling thereof around the limb it encircles. In such an embodiment, the circulation control apparatus 100 is an electro-mechanical traction device that is connected to one or more sensors, such as the aforementioned pressure sensors 116 and a traction mechanism 340 for adjusting the pressure that is applied by the pressure-applying band 102 on the encircled limb.

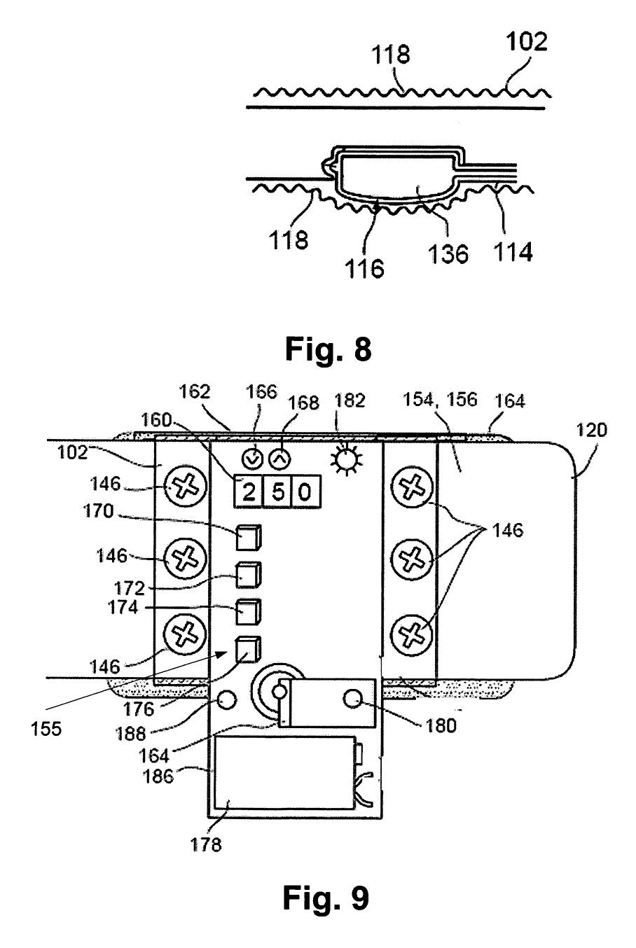

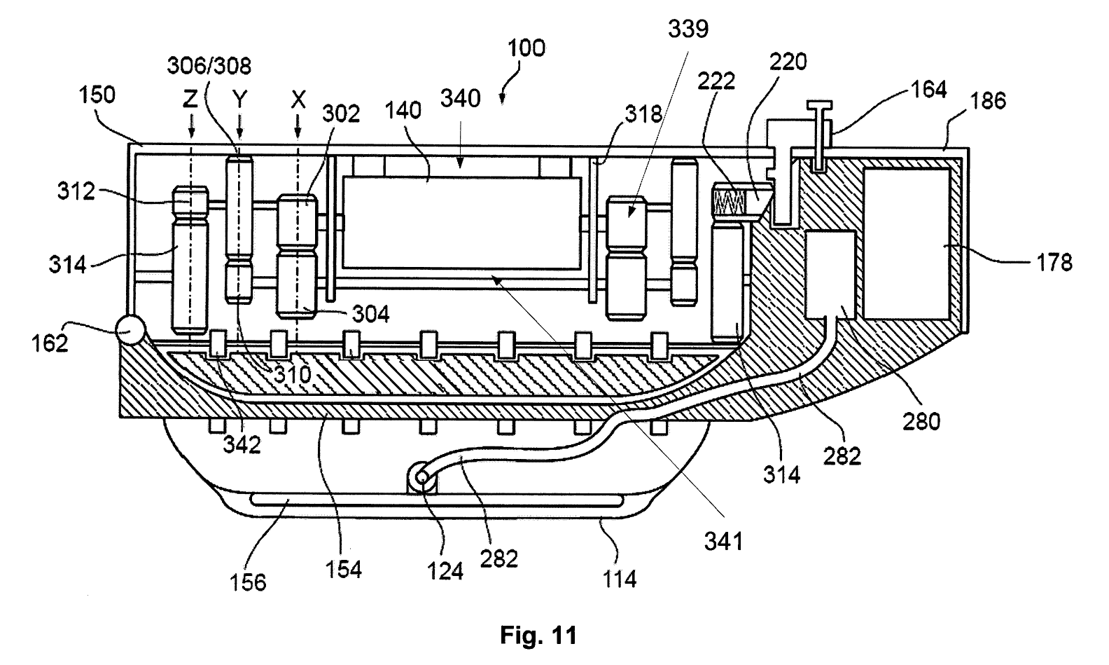

Optionally, the pressure sensors are disposed between the inner side of the pressure-applying band 102, which may be understood herein as the side that in use faces the skin of the occluded limb, and the friction reduction layer 118. Optionally, the one or more grooves 124 are formed along the inner side of the pressure-applying band 102 and allow connecting the pressure sensors 116 to the tension unit 110. Optionally, one or more niches are formed along the inner side of the pressure-applying band 102, allowing the positioning of pressure sensors 116, such as pressure sensors therein. For example, FIG. 8 depicts a sectional schematic illustration of a pressure-applying band 102 that is cut in the middle for creating a niche that allows the positioning of a pressure sensor 116. FIG. 8 depicts an exemplary pressure sensor 116 which is connected to the tension unit 110 via a wire 114 disposed in a groove. The exemplary pressure sensor 116 is disposed in a niche 136 formed on the inner side of the pressure-applying band 102, beneath the friction reduction layer 118. Optionally, the pressure sensors 116 are hosted within the tension unit 110, for example as shown at 280 of FIG. 11, which is a sectional schematic illustration of the tension unit 110, according to some embodiments of the present invention. The pressure sensor 116 is connected via wires and/or tubes 282 connecting through groove 124 in band 102 to the sensing elements 116 in FIG. 11.

As further described below, the traction mechanism 340 is controlled by the pressure evaluating module and designed for pulling and/or releasing the pressure-applying band 102 that encircles the limb. Such a pulling and/or releasing allows the pressure evaluating module to adjust the pressure that is applied by pressure-applying band 102 on the limb according to instructions from the user and/or measurements of the pressure sensors 116.

Optionally, the tension unit 110 comprises a communication unit for sending and/or receiving information from a remote computing unit, such as a laptop and/or a personal digital assistant (PDA) that hosts a pressure-applying module. The communication unit allows sending the measurements of the sensor to the remote computing unit. Optionally, the communication unit comprises a short-range radio interface, such as a Bluetooth™ transceiver, which is defined according to IEEE 802.15.1 specification that is incorporated herein by reference, optionally utilizing a Bluetooth™ enhanced data rate (EDR) chip that is defined according to Bluetooth™ core specification version 2.0+EDR of the Bluetooth™ special interest group (SIG), which is incorporated herein by reference, or a Wibree® transceiver. In such an embodiment, the communication unit may transmit the measurements which are accumulated by the pressure sensors 116 to the remote computing unit.

Reference is now also made to FIG. 9, which is a schematic illustration of the pressure-applying band 102 and tension unit 110 of FIG. 4 from the top, according to some embodiments of the present invention. In the embodiment that is depicted in FIG. 9, the tension unit 110 is connected to a feedback mechanism, such as a user interface (UI) 155, that includes one or more presentation units, such as one or more light emitting diodes (LEDs) 182, a liquid crystal display (LCD) display 160, a speaker 188 and/or any combination thereof, as further described below.

As described above, the pressure evaluating module is connected to the pressure sensors and to the traction mechanism 340 that is used for pulling and or realizing the pressure-applying band 102 to adjust the pressure that is applied on the limb. In such an embodiment, the pressure evaluating module may be used for actuating the traction mechanism 340 to apply a desired level of pressure on the limb. Optionally, the pressure evaluating module instruct the traction mechanism 340 to pull and/or to release the pressure-applying band 102 until the pressure sensors indicate that a desired level of pressure is applied on the limb. Optionally, the patient and/or the caretaker are designed to use the UI 155 for adjusting the desired level of pressure. Optionally, the UI 155 is used for allowing the user to input medical information about the patient. As used herein, medical information means, inter alia, limb dimensions and information that is related to the patient, such as an age group, a gender, medical condition, a blood pressure, patient history, and sensitivities. Optionally, the pressure evaluating module is designed to calculate a desired level of pressure according to the inputted medical information. Optionally, the pressure evaluating module is connected to a repository, such as a memory element, for example a flash memory, that host a plurality of pulling patterns, each associated with a different patient prototype. In use, the pressure evaluating module identifies a match between one of the different patient prototypes and the inputted medical information and uses the respective pulling pattern to instruct the traction mechanism 340.

In some embodiments of the present invention, the tension unit 110 comprises a timer module (not shown) for measuring a pressure period during which the sensors 116 pressure is applied by the pressure-applying band 102. The pressure period may be used for evaluating a desired level of pressure and/or alarming a patient and/or a caretaker according to the measurements of the sensors 116. Optionally, the timer module is connected to a presentation unit, such as a display, for example as shown at 160, for indicating to a patient and/or a caretaker a period during which a period has been applied, by the pressure-applying band 102, circumferentially upon the skin and underlying tissues of a limb. Optionally, the timer module may be connected to the UI 155 that allows the outputting of an audible alarm and/or a visual alarm when the traced period passed the safe occlusion period.

Optionally, the timer module is used for alarming the patient and/or the caretaker when a safe occlusion period has passed. The timer module is optionally activated by the patient and/or the caretaker, for example using the UI 155. The UI 155 may be used for adjusting a pre-set duration that defines the safe occlusion period. Optionally, the UI 155 stores a number of safe occlusion periods, each defined to a patient with different medical condition and/or history. In such an embodiment, the user uses the UI 155 to selected one of the number of safe occlusion periods and the pressure evaluating module is designed for instructing the traction mechanism to adjust the pressure that is applied on the limb accordingly. Optionally, the safe occlusion period is calculated according to the time that elapses since the activation of the timer and/or according to the pressure which applied by the pressure-applying band 102. Optionally, the safe occlusion period is calculated dynamically according to the pressure that has been applied by the pressure-applying band 102 at any given moment during the elapsed time. The timer module is optionally activated using a sensor for deducting the presence of the pressure-applying band 102, for example according to measurements of the pressure sensors 116 and/or a pressure detector that is designed to activate the timer module when a pressure above a certain threshold is applied.

In some embodiments of the present invention, the pressure evaluating module of the tension unit 110 is connected to the aforementioned communication unit. Optionally, the pressure evaluating module may instruct the communication unit to transmit warning signals to the communicating computing unit. Such signals may be send of pressure is applied for a period that is longer than the safe occlusion period.

Optionally, the pressure evaluating module may instruct the communication unit to transmit a report every predefined period. Optionally, a central computing unit is designed to receive reports and/or warning signals from a number of different signals from different circulation control apparatuses, such as shown at 100. Such a central computing unit may assist in managing a complicated situation where many casualties are involved.

In some embodiments of the present invention, the pressure evaluating module of the tension unit 110 is connected to a memory element, such as a random access memory (RAM) and/or flash memory, and/or a drive for reading memory cards, such as flash card. The memory element and/or card may be used to store records of information which is related to the operation of the circulation control apparatus 100, for example the pressures and/or the durations which have been measured by the pressure sensors 116 and/or timer module, including the time of the day and/or the date. Optionally, the pressure evaluating module is connected to the display of the UI 155 that allows the caretaker and/or the patient to access records which are hosted in the memory element and/or the memory card. Optionally, the pressure evaluating module is connected to the aforementioned communication unit and allows the transmitting of accumulated data to a remote computing unit, optionally as described above.

As described above, the UI 155 optionally comprises a digital display 160, such as a LCD display. The UI 155 may be used by the tension unit to indicate the pressure that is measured by the pressure sensors 116, for example in pressure units such millimeter (mm)/hectogram (Hg) and optionally a desired duration of an occlusion activation, for example as described above.

As described above, the UI 155 may include a set of buttons, for example as shown at 170, 172, 174, and 176 that allows the patient and/or the caretaker to control the tension unit 110. Optionally, one of the buttons is a SET PRESSURE button 170 that may be used for adjusting a required pressure. Optionally, the UI 155 includes two arrow buttons 166 and 168 for increasing and decreasing the required pressure, respectively. Similarly, the UI 155 optionally includes a SET TIME button 172 for allowing a user to set and/or to adjust the safe occlusion period by using the arrow buttons 166 and 168. Optionally, one of the buttons is an alarm silence button 174 for stopping the audible and/or visible alarm that is produced by the timer module.

Optionally, the tension unit 110 includes a battery housing 186 for connecting one or more batteries 178 for supplying power to the circulation control apparatus 100. The batteries 178, which may be alkaline batteries, are selected to facilitate sterilization by Ethylene Oxide (ETO), or other means of the entire system. Optionally, the battery 178 drives the motor drive 140, which, in turn, turns the gears of the traction mechanism 340, optionally as described above. Optionally, the UI 155 includes a LOW BATTERY indicator 180, such as a light emitting diode (LED), to indicate when the power of the batteries 178 is under a pre-set level of voltage or charge.

As described above, the traction mechanism 340 is designed to pull the pressure-applying band 102 in order to apply pressure on the limb, as further described above. Optionally, the traction mechanism 340 includes a fast release mechanism for allowing the patient and/or the caretaker to release quickly the applied pressure. Optionally, the traction mechanism 340 includes a pull handle 182 that is connected to a ratchet mechanism that is engaged with the gearwheels of the traction mechanism 340, for example as depicted in numeral 130 of FIG. 14. The pulling of the pull disengages the ratchet mechanism and allows the pressure-applying band 102 to slide against the pulling direction.

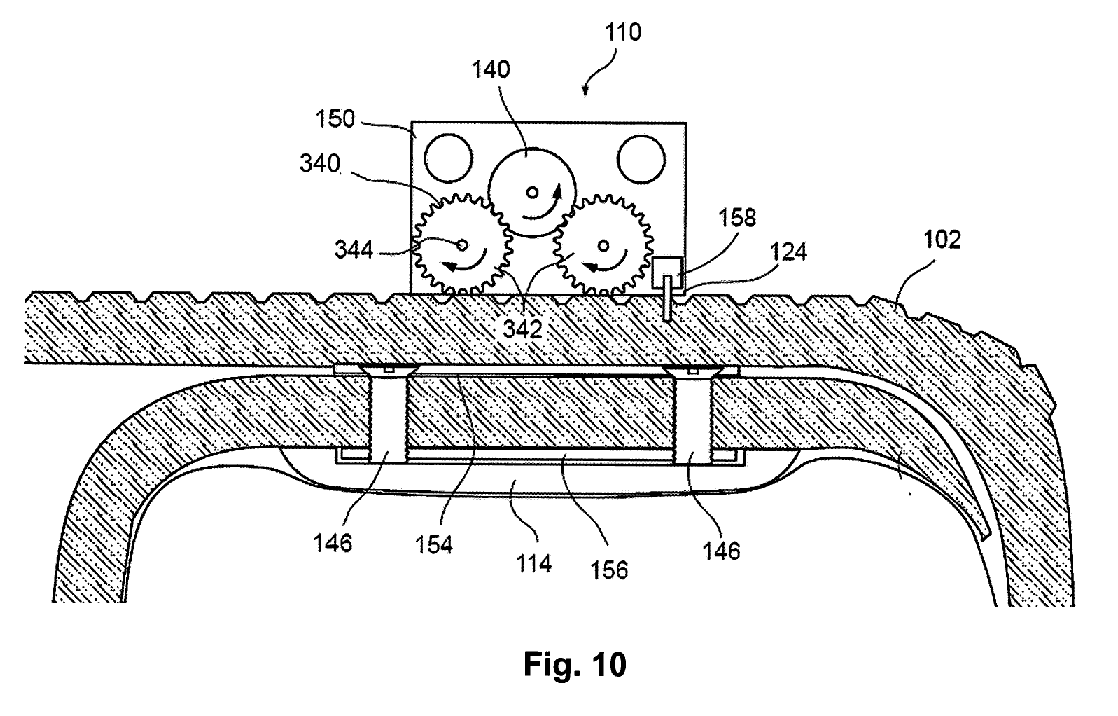

Reference is now also made to FIG. 10, which is a sectional schematic illustration of the tension unit 110 and the pressure-applying band 102 which are depicted in FIG. 9, according to some embodiments of the present invention.

Optionally, the tension unit 110 is secured to the pressure-applying band 102. Optionally screws 146 are used for connecting between two rigid compression plates 154, 156, such as stainless steel plates, which are used for firmly attach the tension unit 110 to the pressure-applying band 102.

As described above, the traction mechanism 340 includes a presence sensor 158, such as an optical, mechanical, and/or electromagnetic sensor that allows the detection of the pressure-applying band 102. Optionally, the presence sensor 158 includes a tip that is pressed whenever the pressure-applying band 102 is positioned in proximity thereto. Optionally, the traction mechanism 340 is operated by the caretaker and/or the patient. In such an embodiment, the caretaker and/or the patient may press an activate push button, for example as depicted in 170 of FIG. 9, for initializing the pulling of the pressure-applying band 102.

FIG. 10 further depicts an exemplary traction mechanism 340 from the side. The exemplary traction mechanism 340 includes a set of gearwheels that may be engaged with the pressure-applying band 102. Optionally, the gears and ratchets of the traction mechanism 340 are hosted in a housing 150 that part of the tension unit 110.

Optionally, in order to facilitate the sliding of the pressure-applying band 102 into the traction mechanism 340 for allowing the pulling thereof in a manner that allows occlusion of the limb, as further descried above, the tip 122 of the pressure-applying band 102 is cut in a curved or pointed shape, for example as shown at FIG. 4. Such a tip allows the patient and/or the caretaker to slide, accurately and easily, the pressure-applying band 102 into a feeding opening 124 of the tension unit 102. In such a manner, a wounded and/or an unskilled patient and/or caretaker may easily use the circulation control apparatus 100 to slow or prevent blood loss. First, the unskilled patient and/or caretaker may easily position pressure-applying band 102 around an injured limb, for example using only one hand, while its tip is in front of the feeding opening. Then, the traction mechanism 340 automatically pulls the pressure-applying band 102 until it applies circumferential pressure, optionally in a desired intensity, on the skin and the underlying tissues of the limb. Such a pulling completes the occlusion of the limb and allows the patient and/or the caretaker to slow or prevent blood loss and/or to prepare the limb for a medical operation. It should be noted that in such an embodiment the patient and/or the caretaker do not have to be trained to provide medical treatment as the circulation control apparatus 100 is designed control the applied pressure in an automatic manner.

Reference is now also made to FIG. 11, which is a sectional schematic illustration of gears and ratchets of exemplary traction mechanism 340, according to some embodiments of the present invention. FIG. 11 depicts a hinge 162 for supporting the opening of the housing 150 and a locking lever 164 that catches a spring 222, supported by a latch 220, to prevent an inadvertent opening of the housing 150. FIG. 9 depicts the traction mechanism 340 in a locked position. The releasing of the locking lever 164 allows a quick release of the pressure-applying band 102 from the tension unit 110 thereby facilitating a rapid release of the circumferential pressure that is applied to the limb by the circulation control apparatus 100.

As described above, in some embodiments of the present invention, the tension unit 102 is designed to pull and/or to release the pressure-applying band 102 in order to allow the occlusion of the limb. In use, a motor drive 140 is activated to actuate a set of gears. The motor drive 140 increases the transmission torque and rotates the gearwheels of the traction mechanism 340 around their axes. Optionally, the motor drive 140 actuates two parallel gearwheels 302, 339 for rotating two traction gearwheels 342 which are used simultaneously for pulling the pressure-applying band 102. Then, the motorized traction continues until the pressure sensor 118 detects a certain pressure on the limb and/or on the pressure-applying band 102. As described above, after a certain pressure is attained, the timer module may be activated to monitor the time occlusion period.

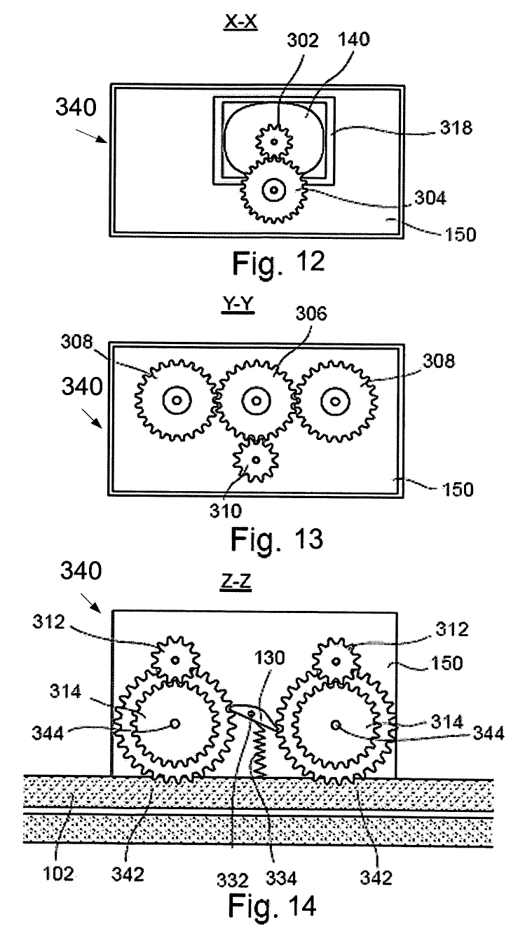

In order to clarify the following description, reference is now also made to FIGS. 12-14, which are three crosscuts of transmission segments of the traction mechanism 340 that is depicted in FIG. 11, according to some embodiments of the present invention. FIGS. 12-14 are respectively marked as axes X, Y, and Z in FIG. 11.

The set of gearwheels of the traction mechanism 340 is optionally designed to reduce the pulling speed and to increase the torque of the motor drive 140 in a manner that increases the utility of the power that is provided by the batteries of the circulation control apparatus 100. The following description discloses an exemplary set of gearwheels that provides a torque-speed conversion from a higher speed motor to a slower but more forceful output that is designed for pulling the pressure-applying band 102. The following description refers to the transmission of the gearwheels at the left side of the figure however respectively describes the transmission of the gearwheels at the right side of the figure.

The motor drive 140 is fixed firmly in a designated frame, for example as shown as 318. As shown at FIG. 12, the shaft of the motor drive 140 actuates an upper central gearwheel 302 that is disposed thereon. The upper central gearwheel 302 is engaged with a larger lower central gearwheel 304 that is connected to a lower shaft 341. As shown at FIG. 13, the lower shaft 341 actuates a small lower central gearwheel 310 that is engaged with a large central upper gearwheel 306. The large central upper gearwheel 306 is engaged with left and right upper gearwheels, as shown 308. The left and right upper gearwheels 308 are respectively connected to left and right shafts 344, which respectively actuate, as shown at FIG. 14, small upper left and right gears 312. The small upper left and right gears 312 are respectively engaged with left and right large lower gearwheels 314. Each one of the left and right shafts 344 are threaded in the center of a traction gearwheel 342. The traction gearwheels 342 are used for advancing the pressure-applying band 102 beneath them by the rotation thereof. The traction mechanism 340 further comprises a ratchet mechanism 130 that includes a spring 334 and a hinge 332. The hinge 332 is engaged with the left and right traction gearwheels 342 so it rotates in one direction to permits the rotational motion of the traction gearwheels 342, while the spring 134 prevents the hinge 332 to disengage from the traction gearwheels 342 thereby stops the traction gearwheels 342 from rotating in the other direction. In such a manner, the pressure-applying band 102 may advance however cannot slip back as long as the hinge 332 is engaged.

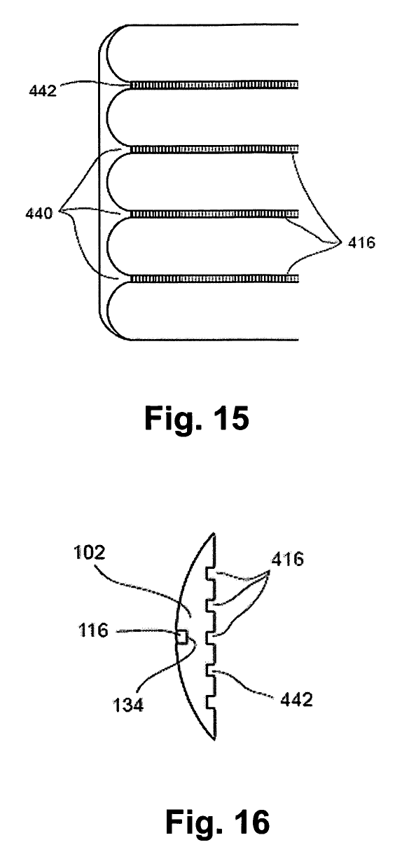

Optionally, in order to improve the traction of the traction gearwheels 342, the upper side of the pressure-applying band 102 has longitudinal grooves therealong, for example as described in FIG. 15, which is a schematic illustration of a section of the pressure-applying band 102 from above, according to some embodiments of the present invention. The longitudinal grooves 416 may be serrated and/or teethed 442 to support the integration of the teeth of the gearwheels therewith. The longitudinal grooves 416 prevent or reduce the slipping of the traction gearwheels 342 and/or pressure-applying band 102 during the traction process. Optionally, in order to ease the initiation of the traction action and the engaging between the longitudinal grooves 416 and the traction gearwheels 342, each one of the longitudinal grooves 416 is formed with entrance funnel 440 at the tip of the pressure-applying band 102. For example, FIG. 16 is a schematic illustration of a crosscut of the pressure-applying band 102 with the aforementioned longitudinal grooves 416.

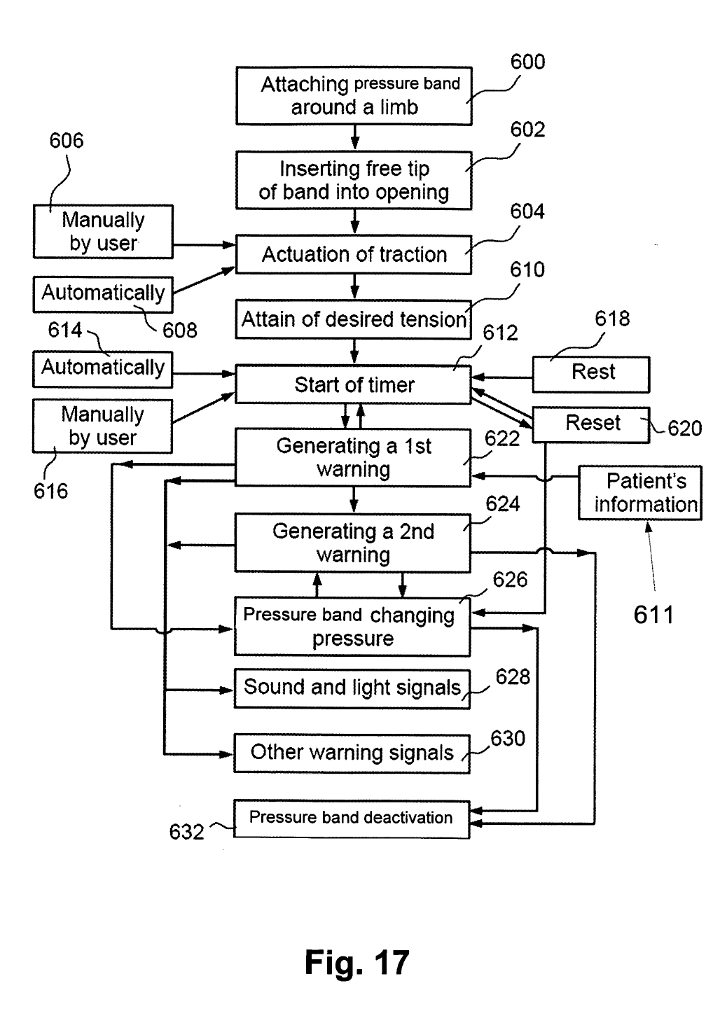

Reference is now made, once again, to FIG. 4, FIG. 10 and to FIG. 17, which is a schematic illustration of a method for controlling the flow of blood of arteries and/or veins of a limb, according to some embodiments of the present invention. As depicted in FIG. 17, in use, the pressure-applying band 102 is drawn 600 around a limb. When the tip 122 of pressure-applying band 102 is inserted 602 into the opening 124 of electromechanical traction device 110, the traction 604 is actuated manually 606 by a user by pressing an ON button, for example as shown at numeral 176 of FIG. 9. Alternatively, the traction 604 may be actuated automatically 608 by an optical, mechanical, and/or electromagnetic sensing element 158, for example as shown in FIG. 10. The traction mechanism 340 than pulls the pressure-applying band 102 until the pressure or the tension which is measured by the pressure sensors 116, as described above, attains 610 a certain level.

Optionally, the timer module is activated 612 for verifying that the pressure of the circulation control apparatus 100 is not applied for more than the safe occlusion period. As described above, the timer module may be activated automatically 614 and/or manually 616. Optionally, it is possible to hold 618 and/or to reset 620 the safe occlusion period. Optionally, the timer module is designed for generating different warnings for alarming the patient and/or the caretaker in more than one situations and/or according the severity of estimated medical condition of the limb and or the patient. The generation of the alarms may be adjusted according to medical information about the patient 611, which is optionally obtained as described above. As described above, the tension unit 110 is designed to adjust the pressure that is applied by the pressure-applying band 102, optionally automatically according to the measurements of the pressure sensors 116. Optionally, as depicted in FIG. 17, the timer module generates a first signal 622 when the measurements indicate that the condition of the patient is in danger and generates a second signal 624 when the pressure-applying period has reached the safe occlusion period. These signals are applied to control the operation of the traction mechanism 340 that pulls and/or releases the pressure-applying band 102 accordingly. Now, as shown at 626, the traction mechanism 340 releases the pressure-applying band 102 until the pressure or the tension that is measured by the pressure sensors 116, as described above, attains a certain level.

In some embodiments of the present invention, these signals are used for instructing a presentation unit, such as a display and/or a speaker, to present a visible alarm and/or an audible alarm 628 and/or any other alarm 630, such as a vibration alarm. For clarity, the alarm may be activated by a respective signal.

Optionally, if the pressure is applied for a period that is longer than the safe occlusion period, the timer module optionally generates a signal that instructs the traction mechanism 340 to release the pressure-applying band 102, thereby to reduce the pressure that is applied by the pressure-applying band 102, for example as shown at 632 of FIG. 17. For example, when the traction mechanism 340 that is depicted in FIGS. 11 and 12-14 is used, the traction mechanism 340 releases the pressure-applying band 102 by disengaging the hinge 332 from the traction gearwheels 342 thereby allowing them to rotate in both directions.

In some embodiments of the present invention, the generating of the these signals is accompanied with sound and light signals 628, wireless transmission of detailed signals 630, such as a transmission using the aforementioned communication unit.

In some embodiments of the present invention, the sensors and/or the pressure evaluating module are connected to a guidance module that is designed to play instructions for guiding the patient and/or the caretaker during the usage of the circulation control apparatus 100.

It is expected that during the life of a patent maturing from this application many relevant devices and methods will be developed and the scope of the term a device, a sensor, and/or a memory, is intended to include all such new technologies a priori. As used herein the term “about” refers to ±10%.

The terms “comprises”, “comprising”, “includes”, “including”, “having” and their conjugates mean “including but not limited to”.

The term “consisting of “including and limited to”.

As used herein, the singular form “a”, “an” and “the” include plural references unless the context clearly dictates otherwise. For example, the term “a compound” or “at least one compound” may include a plurality of compounds, including mixtures thereof.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. An apparatus for adjusting blood circulation in a limb, comprising:
a pressure applying band, having a central longitudinal axis and parallel lateral edges, configured for circumferentially wrapping a portion of the limb and applying a first circumferential pressure beneath said parallel lateral edges and a second circumferential pressure beneath a proximity of said central longitudinal axis;
wherein said first circumferential pressure is lower than said second circumferential pressure.

2. The apparatus of claim 1, wherein said pressure applying band having a non linear cross sectional configuration.

3. The apparatus of claim 2, wherein said non linear cross sectional configuration having a shape selected from a group consisting of a crescent shape, a semi-parabolic shape, a semi-elliptic shape, an elliptic shape, a parabolic shape, or a semicircular shape.

4. The apparatus of claim 2, wherein said non-linear cross sectional configuration is fixed.

5. The apparatus of claim 1, wherein said proximity of said central longitudinal axis having a first elasticity coefficient and said parallel lateral edges having a second elasticity coefficient; wherein said first elasticity coefficient is higher than said second elasticity coefficient.

6. The apparatus of claim 1, further comprising a friction reduction layer between said pressure applying band and the limb, said friction reduction being configured for reducing the friction between the limb and said pressure-applying band.

7. The apparatus of claim 1, further comprising an automatic traction mechanism configured for pulling said pressure applying band to encircle tightly the limb.

8. The apparatus of claim 7, further comprising a pressure adjusting module configured for adjusting said first and second circumferential pressures by activating said traction mechanism according to at least one of a predefined pressure level, a user input defining a pressure level.

9. The apparatus of claim 7, further comprising a pressure adjusting module and at least one pressure sensor configured for measuring at least said second circumferential pressure, said pressure adjusting module being configured for activating said automatic traction mechanism according to said measured second circumferential pressure.

10. The apparatus of claim 1, further comprising a manual traction mechanism configured for allowing a user to pull said pressure applying band around the limb.

11. An apparatus for adjusting blood circulation in a limb, comprising:
a pressure applying band configured for being wrapped around the limb to apply a circumferential pressure thereon;
a traction mechanism configured for pulling said pressure applying band tightly around the limb;
at least one sensor configured for measuring said circumferential pressure; and
a pressure adjusting module configured for activating said traction mechanism according to said measured applied pressure for adjusting said circumferential pressure.

12. The apparatus of claim 11, further comprising a presentation unit for presenting said measured circumferential pressure.

13. The apparatus of claim 11, further comprising a user interface for allowing a user to input a requested circumferential pressure, said pressure adjusting module being configured for activating said traction mechanism according to said requested circumferential pressure.

14. The apparatus of claim 11, wherein said at least one sensor comprises at least one of a pressure detection element for measuring said circumferential pressure and a tension detection element for detection a tension level of said pressure applying band.

15. The apparatus of claim 14, wherein said pressure detection element is a member of a group consisting of a linear variable differential transformer (LVDT), a linear potentiometer, and a pressure transducer.

16. The apparatus of claim 11, further comprising a timer module configured for timing a period during which said pressure applying band apply said circumferential pressure around the limb.

17. The apparatus of claim 16, further comprising an alarming unit for alarming a user according to said timed period.

18. The apparatus of claim 13, further comprising a memory element for storing a plurality of pulling patterns, said user interface being configured for allowing said user to select one of said plurality of pulling patterns, said requested circumferential pressure being adjusted according to said selected pulling pattern.

19. The apparatus of claim 18, wherein each said pulling pattern is defined according to medical information of a patient, said medical information being selected from a group consisting of an age group, a gender, a medical condition, a blood pressure, patient history, and sensitivities.

20. The apparatus of claim 11, wherein said traction mechanism having a feeding opening for supporting a positioning of said pressure applying band to allow said pulling, further comprising a band detection sensor for detecting a presence of said pressure applying band in a proximity to said feeding opening, said pressure adjusting module being configured for activating said traction mechanism according to said detection.

21. The apparatus of claim 20, wherein said pressure applying band has a corrugated tip for facilitating said pulling.

22. The apparatus of claim 11, further comprising a communication unit for transmitting said measured circumferential pressure to a remote computing unit.

23. The apparatus of claim 16, further comprising a memory element for recording said period.

24. The apparatus of claim 11, wherein said traction mechanism comprises at least one traction gearwheel, said traction mechanism being configured for actuating said at least one traction gearwheel for allowing said pulling, said pressure applying band having at least one groove for increasing the friction between said at least one traction gearwheel and said pressure applying band.

25. The apparatus of claim 11, further comprising a quick-release mechanism configured for allowing a prompt reduction of said circumferential pressure.

26. A method for adjusting blood circulation in a limb, comprising:
providing a required circumferential pressure;
allowing a user to encircle a pressure applying band around a limb;
detecting said encircling; and
mechanically adjusting said pressure applying band to apply said required circumferential pressure around said limb.

27. The method of claim 26, wherein said mechanically adjusting comprises pulling said pressure applying band while sensing said circumferential pressure; wherein said pulling being stopped according to a ratio between said sensed circumferential pressure and said required circumferential pressure.
